(19) United States
(12) Patent Application Publication
(10) Pub. No.: US 2004/0001786 A1
(43) Pub. Date: Jan. 1, 2004

(54) Anion-generating device

(76) Inventor: Te-Chin Jan, Taipei (TW)

Correspondence Address: BRUCE H. TROXELL, SUITE 1404, 5205 LEESBURG PIKE, FALLS CHURCH, VA, 22041, US

(21) Appl. No.: 10/180,035

(22) Filed: Jun. 27, 2002

(51) Int. Cl. A62B 7/08; B01J 19/08; A61L 9/00

(52) U.S. Cl. 422/306; 422/305; 422/121; 422/123; 422/186; 422/907

(57) ABSTRACT

The invention is to provide an anion-generating device, which may generate anion to the air, and which may design the air ionization device as a modular structure. Further, when there is a short circuit occurred in the interior circuit of the anion-generating device, it may automatically switch off the high-voltage power source for protecting the anion-generating device. By the arrangement of a switch device, the anion-generating device is resumed to normal operation after being automatically switched off. The invention controls and generates the necessary high-voltage power source by designing a controlling device, which includes a logic controlling circuit and a high-voltage power-source generating circuit, wherein the logic controlling circuit is then comprised of a switch and an indicating device, which may indicate the operational state, while the high-voltage power-source generating circuit may generate high-voltage power source to ionize the air.

### FIELD OF THE INVENTION

The invention relates to an anion-generating device, particularly to a kind of air ionization device that is designed as a modular structure for facilitating easy change or cleanness and, when there is a short circuit occurred in the interior circuit, it may be automatically switched off to protect the anion-generating device and, by the arrangement of a switch device, the anion-generating device is made to resume normal operation after being automatically switched off.

### BACKGROUND OF THE INVENTION

Accordingly, the structure of a common anion-generating device applies the principle of potential difference to generate an electric-dizzy discharge with high voltage. Applying the ionized metal thread in the anion-generating device, the air particle is ionized, charged, become anion-typed, and then dispersed to the domestic space, such that the effect of the air to contain more anion component is fulfilled.

However, when the air enters the cleaner, most dust and impurity in the air will follow the air to flow into the cleaner, such that the ionized thread in the cleaner is easily broken up because of oxidation, or the electricity conductive plate is oxidized to influence the effect of electricity conduction, so that it is impossible to further generate enough anion. Furthermore, when the dust in the electricity conductive plate is accumulated to a certain level or, because there are too many dusts accumulated in the electricity conductive plate and the ionized thread or, because insects (e.g., mosquito and fly) enter the interior of the anion-generating device to cause the disasters of fire, electric leakage, or short circuit, etc., the normal operation of the anion-generating device is influenced, the efficiency is decreased, and many out-of-works are happened.

Therefore, the user must open the interior of the anion-generating device regularly and, after taking apart the device, both the electricity conductive plate and the ionized thread are then cleaned and maintained. Not only is the cleaning procedure tedious, but also is it easy to damage the interior structure of the anion-generating device during the taking-apart procedure because of carelessness. Particularly, when there are objects intruding the interior of the anion-generating device, it is usually incurred the damage caused by the short circuit of the anion-generating device and, it is also too late to open the machine for cleanness at this time.

Therefore, if the shortcomings of the prior arts described therein before can be improved, then the unsolved problem of the prior arts can be solved effectively and, that is why the invention is an innovative breakthrough in this technique range.

### SUMMARY OF THE INVENTION

According to this, in order to improve the shortcomings of the prior arts, the inventor, through a long time of devotion, research, and experiment, has finally designed an anion-generating device of the invention, of which the main objective is to arrange an ion-generating module located at the position of the adjacency of the air inlet to charge and ionize the dust of the air that is sucked into the cleaner, wherein the anion-generating module arranged internally generates ion wind and is designed as a modular structure for facilitating cleanness and maintenance, such that the usage life of the anion-generating device may be further prolonged.

A further objective of the invention is to design a controlling device in the interior of the anion-generating device for controlling the internal circuit and the action of the anion-generating module wherein, when the short circuit is happened, the action of the anion-generating device is stopped to prevent it from damaging and, by the arrangement of a switch device, the operational state is resumed and, by the design of an indicating device, the user will thereby understand the operational state in the anion-generating device.

An another further objective of the invention is to generate anions with high-voltage power source and exhaust the anions into the domestic circumstance in company with the clean air, such that the anion amount in the air is increased and it is beneficial to human body. Wherein, the ion-generating module is an electricity conductive piece of high-voltage power source structured as a saw-tooth type and is constructed by two parallel electricity conductive plates for adsorbing ion wind.

The invention is comprised of a front shell body, a rear shell body, an upper lid, a lower shell body, an ion-generating module, and a controlling device, wherein the front shell body has an exhaust outlet and the rear shell body has an air inlet, which is applied for sucking the outside air into the interior of the anion-generating device, while the exhaust outlet is applied for sending the air that is generated with anions from the interior of the anion-generating device to the outside circumstance, and the upper lid may be opened for taking out or changing the connected ion-generating module; that is, the upper lid and the ion-generating module are designed as one body, such that the user may easily clean and maintain the electricity conductive piece installed in the ion-generating module.

The controlling device is comprised of a logic controlling circuit and a high-voltage power-source generating circuit, wherein the logic controlling device may control the electric action of the anion-generating device and may automatically switch off the action of the air ionization operated by the ion-generating module when there is a short circuit occurred in the first electrode portion and the second electrode portion, while the high-voltage power-source generating circuit is connected to the logic controlling circuit and receives the control from the logic controlling circuit for generating the necessary high-voltage power source for the first electrode portion.

For your esteemed members of reviewing committee to further recognize and understand the objectives, the shapes, the characteristics, the functions, and structures, etc. of the present invention, a detailed description in matching with corresponding drawings is presented thereinafter.

### BRIEF DESCRIPTION OF THE DRAWINGS

FIG. 1 is a preferred embodiment for the three-dimensional illustration of the invention.

FIG. 2 is a preferred embodiment for the front view of the invention.

FIG. 3 is a preferred embodiment for the rear view of the invention.

FIG. 4 is a preferred embodiment for a three-dimensional explosive illustration of the invention.

FIG. 5 is a preferred embodiment for a block diagram for an interior circuit of the invention.

FIG. 6 is a preferred embodiment for an illustration for the ion-generating module of the invention.

FIG. 7 is a preferred embodiment for an ion-generating illustration of the invention.

FIG. 8 is a preferred embodiment for a circuit drawing of the logic controlling circuit of the invention.

FIG. 9 is a preferred embodiment for a circuit drawing of the high-voltage power-source generating circuit of the invention.

### DETAILED DESCRIPTION OF THE INVENTION

The invention is to provide an anion-generating device, which may design the air ionization device as a modular structure for facilitating the user to easily take it out for change or cleanness, such that the usage life of the anion-generating device is prolonged and, when there is a short circuit occurred in the interior circuit of the anion-generating device, the interior high-voltage power source may be automatically switched off for protecting the anion-generation device and, by the arrangement of the switch device, the anion-generating device may be resumed to normal operation after being automatically switched off.

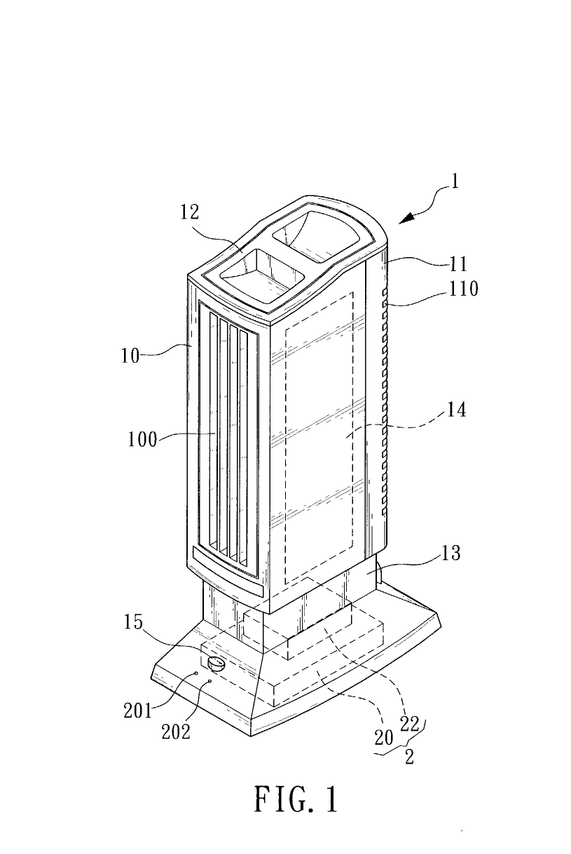

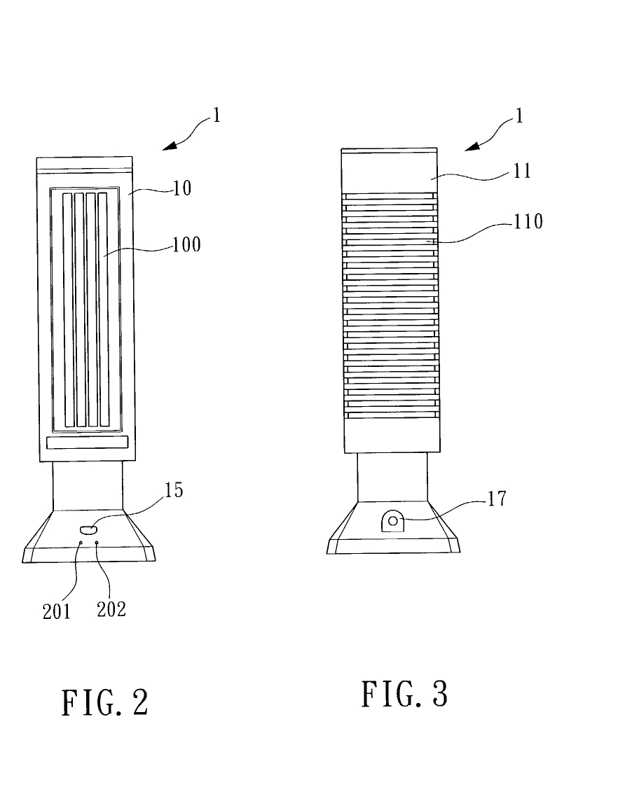

Please refer FIG. 1 to FIG. 3, wherein FIG. 1 is a preferred embodiment of the three-dimensional illustration of the invention, and FIG. 2 and FIG. 3 are the front view and the rear view of the invention respectively. As shown in FIG. 1, the external portion of the anion-generating device 1 according to the invention is a shell structure, which is further comprised of a front shell body 10, a rear shell body 11, a lower shell body 13 that may provide a support for the anion-generating device 1, an upper lid 12, an ion-generating module 14, and a controlling device 2. The front shell body 10 has several exhaust outlets 100, while the rear shell body 11 has several air inlets 110, wherein the air inlet 110 sucks the outside air into the interior of the anion-generating device 1 and the exhaust outlet 100 sends the air that is charged with anion through the interior of the anion-generating device to the outside atmosphere. The upper lid 12 can be opened up to take out the connected ion-generating module 14 for facilitating the user to clean or maintain. Of course, after the cleanness and maintenance, the upper lid 12 together with the ion-generating module 14 are then placed into the interior of the anion-generating device 14, so the upper lid 12 and the ion-generating module 14 may be designed integrally as one body. During cleaning, the user may take out the upper lid 12, and the ion-generating module 14 is also taken out following the upper lid 12 for facilitating the cleanness.

A controlling device 2, arranged in the interior of the anion-generating device 1, may control the entire operational function of the anion-generating device 1 and is comprised of two blocks of circuit substrates. When the anion-generating device 1 is incurred the situation of short circuit, the controlling device 2 may automatically shut off the interior circuit operation of the anion-generating device 1, so the user can actuate the switch 15 to get rid of the phenomenon of short circuit and, at this time, the anion-generating device 1 is still protected under the shutting-off situation and, when the user actuate the switch 15 again, the anion-generating device is resumed to the normal operation. When the anion-generating device is under the situation of normal operation, the switch 15 may make an opening-up or shutting-off action to the anion-generating device; that is, it has the function of power-source control. So, the switch 15 can develop different functions for the anion-generating device 1 under the normal operational state or during the occurrence of a short circuit.

Furthermore, there is an indicating device arranged in the exterior of the anion-generating device 1, such as: light emitting diodes 201, 202, which may indicate the current working situation of the anion-generating device 1, for example, short circuit or non-short-circuit situation. As shown in FIG. 3, a DC power connector 17, connected to a power socket of common family usage, may convert the AC power of family usage into the DC power needed by the anion-generating device.

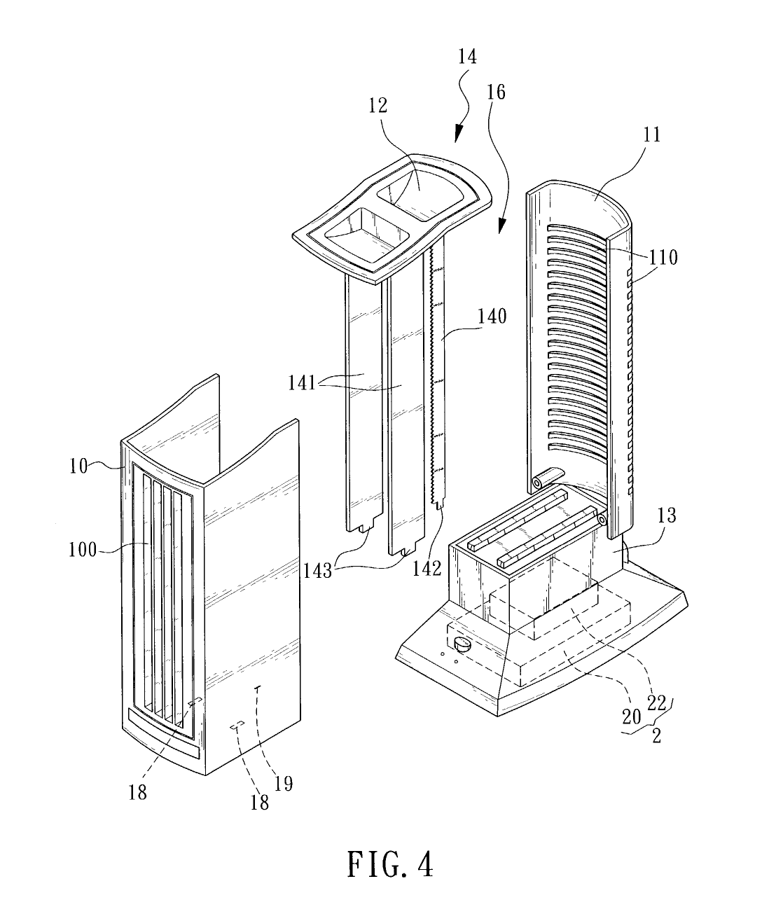

Please refer to FIG. 4, which is a preferred embodiment for the three-dimensional explosive illustration of the invention. The invention includes a shell body that is comprised of a front shell body 10, a rear shell body 11, and a lower shell body 13, wherein the interior of the front shell body 10 and the rear shell body 11 is arranged with an accommodation space 16, which may accommodate the internal circuit device and the ion-generating module 14. The interior of the front shell body 10 is designed with a spring piece 18 and another spring piece 19, which may receive a connection from the connection terminal 142 at the bottom end of the first electrode portion 140 of the anion-generating module 14, and the spring piece 18 may receive a connection from the connection terminal 143 of the second electrode portion 141, while both the sprig pieces 18, 19 will be connected to the high-voltage power source of the high-voltage power-source generating circuit 22. Of course, since the functions of the spring pieces 18, 19 are to provide the connections to the connection terminals 142, 143, so other forms may also be designed for the connection to the terminals 142, 143.

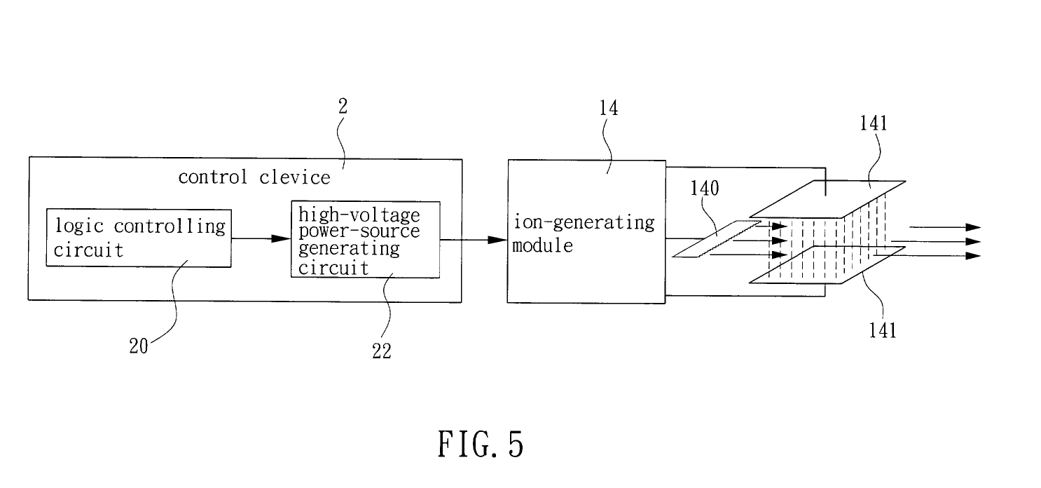

Please refer to FIG. 5, which is a preferred embodiment for the block diagram of the interior circuit of the invention. The interior circuit control of the anion-generating device according to the invention is comprised of a controlling device 2 and an ion-generating module 14, wherein the ion-generating module 14 includes a first electrode portion 140 (i.e., the positive electrode of a high voltage) and a second electrode portion 141 (i.e., the negative electrode of a high voltage). The first electrode portion 140 is connected to the high-voltage power source output from the high-voltage power-source generating circuit 22 and ionizes the air through the operation of point discharge. The second electrode portion 141 is structured as two pieces of electricity conductive plates for adsorbing the dust particles contained in the ion wind. Adsorbing the dust particles contained in the ion wind through the two pieces of the electricity conductive plate of the second electrode portion 141, the impurity contained in the air may be adsorbed naturally and the effect of purification is generated automatically.

The controlling device 2 includes a high-voltage power-source generating circuit 22 and a logic controlling circuit 20, which may control the electric action of the anion-generating device and may shut off the air ionization action operated by the anion-generating module 14 when there is a short circuit occurred in the first electrode 140 and the second electrode 141, while the high-voltage power-source generating circuit 22 is connected to the logic controlling circuit 20 and receives the control from the logic controlling circuit 20 to generate the necessary high-voltage power source for the first electrode 140, so the ion-generating module 14 receives the power source supplied by the high-voltage power-source generating circuit 22, including the positive and negative power source.

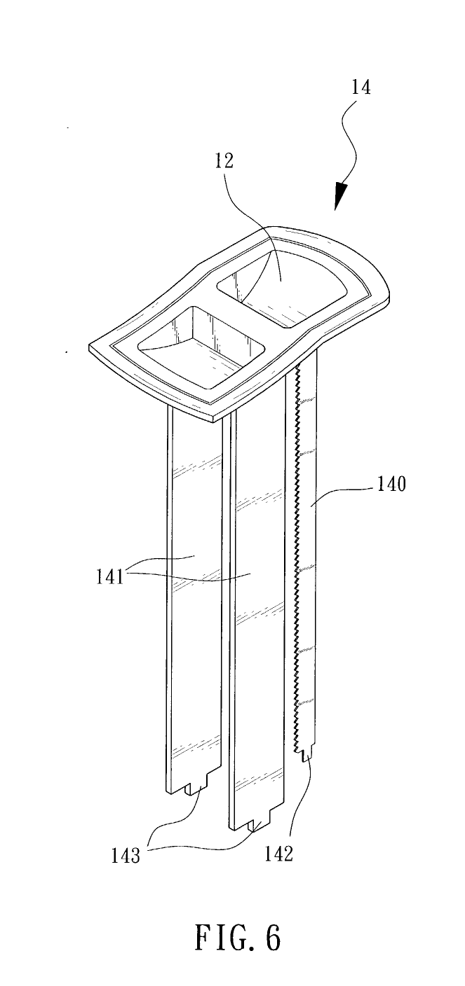

Please refer to FIG. 6, which is a preferred embodiment for the illustration of the ion-generating module of the invention. As shown in FIG. 6, the ion-generating module 14 is one body integrally formed by the first electrode portion 140 (i.e., the positive electrode portion) and the second electrode portion 141 (i.e., the negative electrode portion), such that the ion-generating module 14, by the taking-out manner, may be pulled out or placed into the shell body of the anion-generating device, so the ion-generating module 14 may be designed as a modular frame structure for facilitating to be pulled out from the shell body of the anion-generating device, or be placed into the interior of the shell body. The main point is that the user can easily clean and maintain the positive electrode and the negative electrode. The first electrode portion 140, connected to the positive high-voltage power source, is formed as a saw-tooth type, has several electricity conductive pieces structured as sharp tips, and is located at the adjacency of the air inlet to suck the outside air in and ionize it. The second electrode portion 141, supplied with a negative electrode power source and structured as two pieces of electricity conductive plates, is located at the adjacency of the exhaust outlet to adsorb the air ions coming from the first electrode portion.

When the dust particles are accumulated on the electricity conductive piece of saw-tooth type or on the electricity conductive plates to certain degree or, when they have contacted too long with the air and are oxidized or, because the objects, such as: insects, mosquito, and fly, etc., fly or drop onto the electricity conductive piece of saw-tooth shape or electricity conductive plate, the normal operation of the anion-generating device will be influenced to cause the decrease of efficiency or other dangerous situation of out-of-work so, through the modulization of the ion-generating module, the device will be easy to clean and the generation of these shortcomings can be avoided.

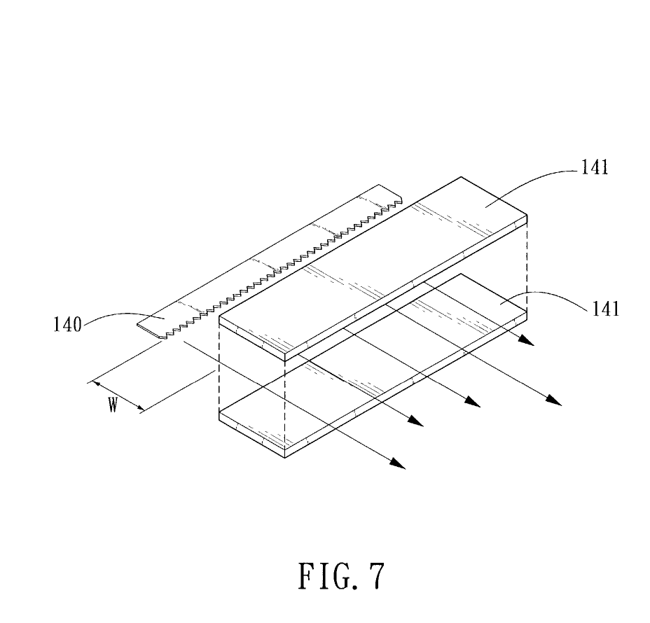

Please refer to FIG. 7, which is a preferred embodiment for the illustration of the ion-generating device of the invention. As shown in FIG. 7, the first electrode portion 140 is kept an appropriate distance w with the second electrode portion 141 and, when the first electrode portion 140 receives a high-voltage power source, its saw-tooth-shaped sharp tips will generate discharge operation, such that the surrounding air is ionized. The second electrode portion 141 is constructed as two pieces of electricity conductive plates 141a, 141b, which will adsorbed the ionized air formed from the air, which surrounds the first electrode 140 and is charged by the point discharge of the first electrode 140, wherein the two pieces of electricity conductive plates constructing the second electrode portion are arranged in parallel, while the first electrode portion 140 is located at the distance w away from the middle plane between the two electricity conductive plates 141a, 141b; that is, when both the electricity conductive plates 141a, 141b are extended backwards certain distance w, they can be parallel with the first electrode portion 140 and, in the meantime, it can be found that the first electrode portion 140 is just located in the middle plane between the extensive positions of the two pieces of the electricity conductive plates 141a, 141b.

The purpose of such kind of design is to form an ion wind that is a directional flow; that is, the air in the outside of the anion-generating device is charged and is carried with anions after entering the air inlet and passing through the ion-generating module 14 and, the air carrying with the discharged anions is then exhausted to the exterior of the anion-generating device to make the anions dispersed in the atmosphere.

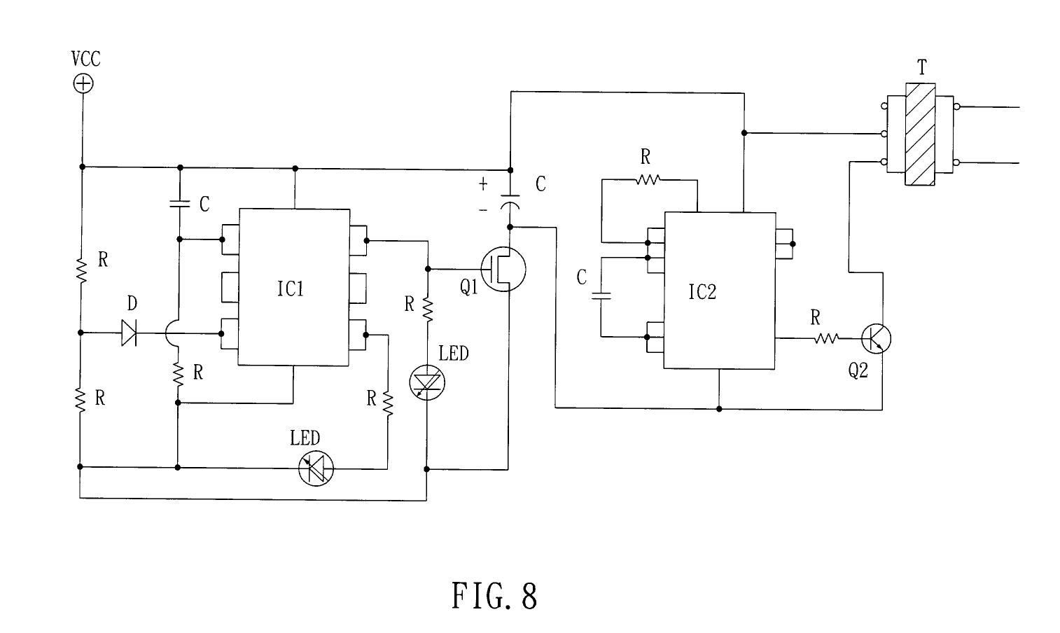

Please refer to FIG. 8, which is a preferred embodiment of the circuit drawing of the logic controlling circuit of the invention. As shown in FIG. 8, the states of the light emitting diodes 201, 202 are controlled by an IC1. For example, when the light emitting diode 201 is illuminated, it is a state of short circuit (i.e., abnormal working state) and, when the light emitting diode 202 is illuminated, it is a non-short-circuit state (i.e., normal working state), while the IC2 may control the output action of the voltage output to the circuit of next level (e.g., the high-voltage power-source generating circuit) by controlling the output voltage through the electric transistor Q2 and the voltage transformer T.

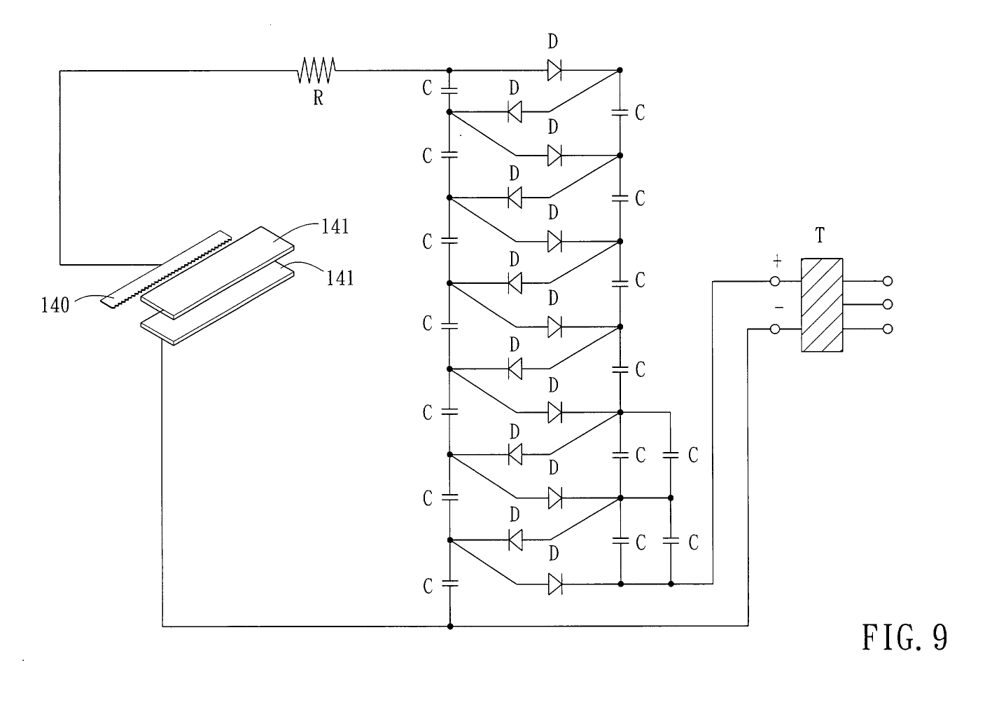

Please refer to FIG. 9, which is a preferred embodiment of the circuit drawing of the high-voltage power-source generating circuit of the invention. As shown in FIG. 9, the voltage signal comes from the FIG. 8 through the output from the voltage transformer T. The double voltage is generated from the series connections and the parallel connections of a double voltage circuit composed of diodes D and capacitance C and, by the double voltage circuit, a positive high-voltage power source needed by the first electrode portion 140 may be generated, while the second electrode portion 141 is connected to the negative terminal of the power source of the voltage transformer T.

Accordingly, the above description is only an illustration for the several preferred embodiments according to the invention. However, the practical application of the invention is not restricted to this description, because any kind of variation that can be easily executed by those who are skilled in this art according to the spiritual range of the invention is all covered by the patent's claims claimed thereinafter.

In sum, the invention has the characteristics of simple structure and easy maintenance. Particularly, when the ion-generating module is needed to clean, because it is designed as a module, the cleaning procedure is very easy; that is, the module may be immersed in the water or scrubbed. The invention not only can clean the air, but also has the effect to increase the anion number in the air. Compared with the products of the prior arts, the invention may indeed prevent the occurrences of short circuit, electricity leakage, or firing, etc., which are caused by the dust particles accumulated in the interior of the anion-generating device. Particularly, when a short circuit is occurred, the anion-generating device will be stopped immediately to avoid any damage, so the shortcoming of the prior arts—because the dust particles are easily accumulated on the surface of the ion-generating device, the ion-generating device is caused to operate abnormally and the expected function can not be fulfilled—can be overcome. Furthermore, the anion-generating device according to the invention, of which structure is simple, the cost is cheap, and the function of anion generation can be developed, and which has the enhancement of particular function.

What is claimed is:

1. An anion-generating device, which includes:
a shell body, in which there is an accommodation space, and on which an air inlet and an exhaust outlet are arranged for facilitating the outside air to enter the device, go through the ionization procedure operated in the accommodation space, and leave the exhaust outlet again;
at least an ion-generating module, which is arranged in the accommodation space, and which includes:
a first electrode portion, which is located at the adjacency of the air inlet and is connected to a high-voltage power source;
a second electrode portion, which includes at least two electricity conductive plates, and which is located at the adjacency of the exhaust outlet, and the ion-generating module may charge and ionize the air particles that are flowed from the first electrode to the second electrode; and
a controlling device, which includes:
a logic controlling circuit, which may control the electric action of the anion-generating device and may automatically shut off the ionization action executed on the air by the ion-generating module when there is a short circuit occurred in the first electrode portion and the second electrode portion;
a high-voltage power-source generating circuit, which is connected to the logic controlling circuit and receives the control coming from the logic controlling circuit to thereby generate a high-voltage power source needed by the first electrode.

2. The anion-generating device according to claim 1, wherein the ion-generating module is a modular frame structure, which includes the first electrode portion and the second electrode portion, and which may be taken out and departed from the shell body or be placed into the shell body.

3. The anion-generating device according to claim 1, wherein the ion-generating module is further comprised of an upper lid, together with which the ion-generating module is designed integrally as one body.

4. The anion-generating device according to claim 1, wherein there is an appropriate space (distance) kept between the first electrode portion and the second electrode portion, such that the ionized air passes through the first electrode portion and flows through the electricity conductive plates constituting the second electrode portion.

5. The anion-generating device according to claim 1, wherein the second electrode portion is constructed as two electricity conductive plates arranged in parallel and it is also parallel to the first electrode portion by extending certain distance toward the first electrode portion to make the first electrode located between the two electricity conductive plates.

6. The anion-generating device according to claim 1, wherein the first electrode portion is a saw-tooth-shaped structure of electricity conductive piece having several sharp tips capable of generating high-voltage discharge.

7. The anion-generating device according to claim 1, wherein there is at least one switch, which is connected to the controlling device, and which may resume the ion-generating device to normal operation (i.e., non-short-circuit situation) after there is a short circuit occurred in the interior of the ion-generating device.

8. The anion-generating device according to claim 1, wherein a first indicating device and a second indicating device are connected to the controlling device respectively, and the first indicating device indicates the interior circuit of the anion-generating device in a short circuit state, while the second indicating device indicates the interior circuit of the anion-generating device in a non-short-circuit state.

9. The anion-generating device according to claim 8, wherein the first indicating device and the second indication device are light emitting diodes (LEDs).

10. The anion-generating device according to claim 1, wherein the logic controlling circuit includes two ICs.

11. The anion-generating device according to claim 1, wherein the logic controlling circuit may be comprised of a circuit substrate, and the high-voltage power-source generating circuit may be comprised of another circuit substrate.

12. An anion-generating device, which includes:
a shell body, in which there is an accommodation space, and on which an air inlet and an exhaust outlet are arranged for facilitating the outside air to enter the device, go through the ionization procedure operated in the accommodation space, and leave the exhaust outlet again;
at least an ion-generating module, which is arranged in the accommodation space, and which includes:
a first electrode portion, which is located at the adjacency of the air inlet and is connected to a high-voltage power source;
a second electrode portion, which includes at least two electricity conductive plates, and which is located at the adjacency of the exhaust outlet, and the ion-generating module may charge and ionize the air particles that are flowed from the first electrode to the second electrode;
it is characterized in that the ion-generating module is a modular structure, which may be taken out or placed into the anion-generating device.

13. The anion-generating device according to claim 12, wherein the ion-generating module is further comprised of an upper lid, together with which the ion-generating module is designed integrally as one body.

14. The anion-generating device according to claim 12, wherein there is an appropriate space (distance) kept between the first electrode portion and the second electrode portion, such that the ionized air passes through the first electrode portion and flows through the electricity conductive plates constituting the second electrode portion.

15. The anion-generating device according to claim 12, wherein the second electrode portion is constructed as two electricity conductive plates arranged in parallel and it is also parallel to the first electrode portion by extending certain distance toward the first electrode portion to make the first electrode located between the two electricity conductive plates.

17. The anion-generating device according to claim 12, wherein it further includes a controlling device, which is comprised of:
a logic controlling circuit, which may control the electric action of the anion-generating device and may automatically shut off the ionization action executed on the air by the ion-generating module when there is a short circuit occurred in the first electrode portion and the second electrode portion;
a high-voltage power-source generating circuit, which is connected to the logic controlling circuit and receives the control coming from the logic controlling circuit to thereby generate a high-voltage power source needed by the first electrode.

18. The anion-generating device according to claim 12, wherein the logic controlling circuit includes two ICs.

19. The anion-generating device according to claim 12, wherein there is at least one switch, which is connected to a controlling device in the anion-generating device, and which may resume the ion-generating device to normal operation (i.e., non-short-circuit situation) after there is a short circuit occurred in the interior of the ion-generating device.

20. The anion-generating device according to claim 12, wherein there is further at least one indicating device, which may indicate the working state of the anion-generating device.

21. The anion-generating device according to claim 12, wherein there are further a first indicating device and a second indicating device, and the first indicating device indicates the interior circuit of the anion-generating device in a short circuit state, while the second indicating device indicates the interior circuit of the anion-generating device in a non-short-circuit state.

22. The anion-generating device according to claim 12, wherein the first indicating device and the second indication device are light emitting diodes (LEDs).

23. The anion-generating device according to claim 1, wherein the logic controlling circuit may be comprised of a circuit substrate, and the high-voltage power-source generating circuit may be comprised of another circuit substrate.
